(19) United States
(12) Patent Application Publication
(10) Pub. No.: US 2021/0085692 A1
(43) Pub. Date: Mar. 25, 2021

(54) AGENTS, COMPOSITIONS AND METHODS FOR TREATING AND PREVENTING ALZHEIMER'S DISEASE

(71) Applicant: UNIVERSITY OF SOUTHERN CALIFORNIA, Los Angeles, CA (US)

(72) Inventors: Roberta Diaz BRINTON, Tucson, AZ (US); Kathleen E. ROGERS, Tucson, AZ (US)

(73) Assignee: University of Southern California, Los Angeles, CA (US)

(21) Appl. No.: 16/980,808

(22) PCT Filed: Mar. 13, 2019
(86) PCT No.: PCT/US2019/022056

§ 371 Date: Sep. 14, 2020

Related U.S. Application Data
(60) Provisional application No. 62/642,360, filed on Mar. 13, 2018.

(51) Int. Cl. A61K 31/57; A61P 25/28

(52) U.S. Cl. CPC A61K 31/57; A61K 9/0019; A61P 25/28

(57) ABSTRACT

Compositions of Allopregnanolone (Allo), and methods of use thereof for treating and preventing Alzheimer's Disease (AD) or dementia, have been developed. In some embodiments, the amount of Allo effective to treat AD or dementia is between about 2 mg and about 10 mg, preferably 4 mg per dose. Methods for identifying subjects for treatment of AD or dementia are also provided. The methods include selecting a subject having one or more Apo E4 gene alleles. Methods of treating a human subject having AD or at risk of AD or dementia are provided. The methods include administering a dosage of from 2 mg to 6 mg to the subject once within a 24 hour period. The dosing is repeated every seven days, or less frequently. The methods stimulate mitosis of neural progenitor cells, stimulate neurite growth and organization, protect against neural loss, or one or more of these neural processes.

### CROSS-REFERENCE TO RELATED APPLICATION

This application claims priority to and the benefit of U.S. Ser. No. 62/642,360 filed Mar. 13, 2018, the disclosure of which is expressly incorporated hereby by reference.

### STATEMENT REGARDING FEDERALLY SPONSORED RESEARCH OR DEVELOPMENT

This invention was made with Government Support under National Institute on Aging grant numbers UF1AG046148, U01AG031115 and U01AG047222. The Government has certain rights in the invention.

### FIELD OF THE INVENTION

This invention is in the field of pharmaceutical compositions for preventing and reversing neurological deficits associated with Alzheimer's disease, and methods of use thereof, particularly compositions containing allopregnanolone.

### BACKGROUND OF THE INVENTION

Alzheimer's disease (AD) is a progressive multifactorial disease, affecting more than 35 million people worldwide, and is the most common dementia of late-life. The mean incidence of AD is 1-3% and is associated with an overall prevalence of 10-30% in persons over 65 years of age which, globally, is predicted to nearly double every 20 years. On average, persons will live with Alzheimer's disease for 10 years. In the US, total costs for caring for the 5 million persons living with the disease is estimated at S200 billion and are projected to rise to S1.1 trillion by 2050. To date, no interventions have demonstrated substantial therapeutic efficacy to prevent, delay or treat AD and several have accelerated disease progression (http://www.alzforum.org/therapeutics). Current thinking in the field embraces the complexity of AD pathophysiology, which has enabled a more diverse therapeutic pipeline targeting multiple aspects of the disease.

Attempts to counteract the effects of acute or neurodegenerative lesions of the brain and/or spinal cord have primarily involved implantation of embryonic neurons in an effort to compensate for lost or deficient neural or neurological function. However, human fetal cell transplantation research is severely restricted. Administration of neurotrophic factors, such as nerve growth factor and insulin-like growth factor, also has been suggested to stimulate neuronal growth within the CNS. A large body of literature explores the potential for neurosteroid-based interventions of AD, for example, Schneider, et al., Arch Neurol 2011; 68:58-66; Carlson, et al. Alzheimers Dement 2011; 7:396-401; Sperling, et al. Lancet Neurol 2012; 11:241-9; Brinton, Nat Rev Endocrinol 2013; 9:241-50; Chen, et al., PLoS One 2011; 6:e24293; Singh, et al. Neurobiol Aging 2011. Wang, et al., Proc Natl Acad Sci USA 2010; 107:6498-503; Wang, et al., J Neurosci 2005; 25:4706-18; 28. Sun, et al. Curr Alzheimer Res 2012; 9:473-80; Lan et al., Hormones and behavior, 1994; 28:537-44; Reddy, et al., the journal of the American Society for Experimental Neuro Therapeutics 2009; 6:392-401; Simon, et al., J Natl Cancer Inst 1997; 89:1138-47; Irwin, et al, Front Endocrinol (Lausanne) 2011; 2:117; Petersen, Nature reviews Drug discovery 2003; 2:646-53; McKhann, et al. Alzheimers Dement 2011; 7:263-9; Green, et al. JAMA 2009; 302:2557-64; Collie, et al., Psychopharmacol 2006; 21:481-8; Falleti, et al., J Clin Exp Neuropsychol 2006; 28:1095-112; Lim, et al. J Clin Exp Neuropsychol 2012; 34:345-58; Bond, et al. Psychol Med 1974; 4:374-80; Sperling, et al., Alzheimer's & Dementia 2011; 7:367-85; Salloway, et al., Neurology 2009; 73:2061-70; and Weiner, et al. the journal of the Alzheimer's Association 2012; 8:S1-68)

However, in spite of significant efforts, to date no satisfactory agents or treatment methods exists to repair, or counteract, the neuronal damage associated with Alzheimer's disease, or the associated cognitive decline or impairment. Accordingly, there is a need for new treatment modalities directed to improving the adverse neurological conditions associated with Alzheimer's disease.

Therefore, it is an object of the invention to provide compositions for the treatment or prevention of neuronal damage associated with Alzheimer's disease and the associated cognitive decline or impairment, and methods of making and using thereof.

### SUMMARY OF THE INVENTION

Allopregnanolone is a first in class regenerative therapeutic for early AD or dementia that targets endogenous neural stem cells and disease modifying mechanisms. Clinical data indicate a favorable safety and tolerability profile, and potential efficacy. Therapeutics to prevent, delay and treat Alzheimer's disease (AD) or dementia represent a regenerative medicine, systems biology approach that target the regenerative system of the brain while simultaneously activating systems to reduce burden of AD pathology. These data extensively characterize mechanisms by which Allo promotes neural stem cell regeneration and restoration of cognitive function

Allopregnanolone (Allo) is a pleiotropic neurosteroid that preclinically promotes neurogenesis and restores cognitive function in AD transgenic models and in wild type aged mice. In addition to neurogenesis, Allo promotes myelin regeneration. Importantly, Allo promotes generation of human neural stem cells in vitro. Simultaneous to promoting regeneration, Allo reduces AD pathology via well-established pathways to decrease the generation of Abeta while also decreasing inflammation. Allo is a low molecular weight neurosteroid endogenous to the brain and blood brain barrier penetrant with abundant existing safety data in animals and humans. The clinical data from use of Allo in persons with MCI or early AD indicate that the regenerative treatment regimen of once per week IV infusion is well tolerated with no indications of Allo-related adverse events. Cognitive testing and extended MRI brain imaging for regenerative surrogate markers were well tolerated and feasible. Safety and tolerability findings in women and men was demonstrated, indicating no adverse outcomes following 24 weeks of once per week Allo exposure at doses exceeding those to be tested in humans.

Compositions for the treatment or prevention of neuronal damage associated with Alzheimer's disease-related decline or impairment or dementia, and methods of making and using thereof have been developed. Compositions contain α-hydroxy-5α-pregnan-20-one (also referred to as allopregnanolone, THP, Allo, or APα), a derivative, analogue or prodrug thereof, a pharmaceutically acceptable salt thereof, or combinations thereof.

In some embodiments, the amount of Allo effective to alleviate one or more symptoms of AD or dementia is between about 2 mg and about 6 mg, preferably 4 mg per dose. Suitable analogues or derivatives of THP include, but are not limited to, 3-beta-phenylethynyl derivatives of 3α-hydroxy-5α-pregnan-20-one; analogues or derivatives of 3α-hydroxy-5α-pregnan-20-one that exhibit substantially equivalent neuro-enhancing activity as 3α-hydroxy-5α-pregnan-20-one; progesterone; and progesterone-like molecules, which are either natural metabolites of progesterone or synthetic variants of progesterone, and exhibit substantially equivalent neuro-enhancing activity as 3α-hydroxy-5α-pregnan-20-one.

Effective therapeutic amounts of the neuro-enhancing agents will depend on the neurological disease or defect being targeted, but generally are between about 2 mg and 10 mg per single dose, preferably between about 3 and 5 mg, more preferably between about 3.5 and about 4.5 mg, most preferably 4 mg per dose, in a human. In one embodiment, the compositions contain about 4 mg of the pharmaceutically active form of 3α-hydroxy-5α-pregnan-20-one or an analogue, derivative, or prodrug thereof.

Methods including selecting a subject who is likely to benefit from treatment with Allo have are also been established. The methods include identifying a subject having one or more biological markers associated with development of one or more of the symptoms of AD or dementia. In a preferred embodiment, the marker is the presence of the Apo-E4 allele.

The compositions can be administered in a single dose within a 24 hour period. Dosing is repeated after a refractory period of about 6 days, about 7 days, or about 8 days, for example, once every week, or less frequently (i.e., dosages are administered more than 6 days, more than 7 days, more than 8 days after the last previous dose). The effective administration periods depend on the particular neurological disease or defect being targeted. Generally, Allo is administered over a period of time of about one month or longer, but can be over about six months, about one year or longer. In a preferred embodiment, 4 mg of allopregnanolone is administered to an adult human with Alzheimers or dementia in a single dose, which is repeated once per week, or less frequently. This dosing regimen maximizes neurogenesis and minimizes pathology burden. FIG. 1 shows the optimal allopregnanolone therapeutic regimen. The compositions are typically administered in single administrations (orally, topically or by injection, with release of the drug into the patient being substantially complete within a 24 hour period) over an extended period of time, for example, at least about 10 weeks, preferably at least about 30 weeks, more preferably at least about 60 weeks, even more preferably at least about 72 weeks, and most preferably as long as the patient is receiving noticeable benefit from the treatment method. In one embodiment, the composition is administered once a week for at least 6 months. The formulation is significantly less effective in these patients if administered more frequently than less than six or seven days between administrations.

Compositions of Allo can be formulated for systemic delivery via enteral or parenteral administration. The compositions can further contain one or more pharmaceutically acceptable excipients, carriers, and/or additives. In some embodiments, allopregnanolone is administered in a transdermal gel containing CARBOMER® 940, and ethanol. In some embodiments, the gel contains from about 2 mg to about 10 mg of bioavailable Allopregnanolone, and is administered once a week or less frequently for a period of at least 3 months, preferably at least 6 months, most preferably for a year or more. Studies showed that the transdermal gel was as effective as subcutaneous administration (0.1% ethanol/phosphate buffered saline). In another embodiment, allopregnanolone is administered via intravenous administration, or intranasally.

In one embodiment, the compositions are administered to enhance neurological function in an individual having one or more symptoms of Alzheimer's disease (AD) or dementia, such as neurological decline or impairment, or decrease in hippocampus tissue volume as assess by MRI. The compositions are administered over a period of time effective to stimulate neural mitosis, to prevent neuronal loss, or combination thereof. Target neurological dysfunctions and disease states include one or more of the symptoms of Alzheimer's disease, such as memory loss and/or reduced learning. In one embodiment, the compositions are administered to reduce β-amyloid accumulation in the brain, which is associated with Alzheimer's disease.

The compositions can also be administered to improve or restore neurological function by inducing or stimulating the generation of new neurons, protecting against neuronal loss, stimulating or inducing neurite outgrowth and organization or protecting against loss of neurites and neural networks, or combination thereof.

### BRIEF DESCRIPTION OF THE DRAWINGS

FIG. 1 is a graphical representation of the optimal allopregnanolone therapeutic regimen.

FIG. 2 is a dot plot showing baseline volume of the left hippocampus (in mm³) versus change in the left hippocampal volume (in mm³) in allopregnanolone cohort 1 (allo cohort 1), allo cohort 2, and placebo groups. F: Female; M: Male; and number indicates age of each participant.

FIG. 3 is a violin plot showing change in the left hippocampal volume (in mm³) in 2 mg, 4 mg, and 6 mg of allopregnanolone, and placebo groups.

FIG. 4 is a violin plot showing change in the left hippocampal volume (in mm³) in 2 mg, 4 mg, and 6 mg of allopregnanolone, and placebo groups in male and female cohorts.

FIG. 5 is Allopregnanolone: Change in Left Hippocampal Volume by APOE Genotype, a dot plot showing baseline volume of the left hippocampus (in mm³) versus change in the left hippocampal volume (in mm³) in allopregnanolone cohort 1 (allo cohort 1), allo cohort 2, and placebo groups by apolipoprotein E (APOE) genotype. 3: APOE3; 4: APOE4; F: Female; M: Male; and number indicates age of each participant.

FIG. 6 is a violin plot showing change in the hippocampal volume (in mm³) in APOE 3, APOE 4, and placebo groups.

FIG. 7 is a violin plot showing change in the hippocampal volume (in mm³) by APOE genotype and gender: in female-APOE3 (F-APOE 3), F-APOE 4, male-APOE3 (M-APOE 3), M-APOE 4, and placebo group.

FIG. 8 is Change in hippocampal volume in Females by dose and APOE status, a violin plot showing change in the hippocampal volume (in mm³) by APOE genotype and allopregnanolone dose in female participants including APOE3/4 with placebo (Placebo-A3/4), APOE3/4 with 2 mg of allopregnanolone (Allo 2 mg-A3/4), APOE3/4 with 4 mg of allopregnanolone (Allo 2 mg-A3/4), APOE3/3 with 6 mg of allopregnanolone (Allo 2 mg-A3/4), and APOE3/4 with 6 mg of allopregnanolone (Allo 2 mg-A3/4).

FIG. 9 is a violin plot showing change in the hippocampal volume (in mm³) by APOE status and allopregnanolone dose in male participants including 2 mg, 4 mg, and 4 mg of allopregnanolone, and placebo groups in APOE4 negative and APOE4 positive participants.

FIG. 10 is iPSC Derived NSCs for Biomarker of Regenerative Responders and Non Responders APOE 4+: Mitochondrial Respiratory Capacity, a dot plot showing percent change in mitochondrial spare capacity in neuronal stem cells (NSC) versus change in the total left hippocampal volume (in mm³) in allopregnanolone cohort 1 (allo cohort 1), allo cohort 2, and placebo groups. F: Female; M: Male; and number indicates age of each participant.

FIG. 11 is a line diagram showing change in oxygen consumption rate (OCR, in pMol/min) in response to ATP coupler, ETC accelerator, Mito inhibitors measured over the period of 100 minutes.

FIG. 12 is a dot plot iPSC Derived NSCs for Biomarker of Regenerative Responders and Non Responders APOE 4+: Mitochondrial Respiratory Capacity, showing percent change in mitochondrial spare capacity in induced pluripotent stem cell (iPSC)-derived neuronal stem cells (NSC) versus change in the total left hippocampal volume (in mm³) in allopregnanolone cohort 1 (allo cohort 1), allo cohort 2, and placebo groups. 3: APOE3; 4: APOE4; F: Female; M: Male; and number indicates age of each participant.

FIG. 13 is a bar graph showing changes from baseline volume of the left total hippocampus in placebo, 2 mg allopregnanolone, and 4 mg allopregnanolone groups after 12 weeks of treatment.

FIG. 14 is a line graph showing changes from baseline volume of the left total hippocampus (in mm³) after 12 week treatment of 4 mg allopregnanolone versus APOE genotype.

FIG. 15 is a graph of the difference in exploration time: Novel versus Familiar, seconds for various groups of animals that were tested.

FIG. 16 of Discrimination Index, ratio, for various groups of animals that were tested.

FIGS. 17A and 17B are graphs of the Differences in Exploration Time: Novel versus Familiar, seconds, for ApoE 3/4F vehicle control and ApoE 3/4F Allo (FIG. 17A) and Discrimination Index, ratio, for ApoE 3/4F vehicle control and ApoE 3/4F Allo (FIG. 17B).

### DETAILED DESCRIPTION OF THE INVENTION

### I. Definitions

The term “analogue”, refers to a chemical compound with a structure similar to that of another (reference compound) but differing from it in respect to a particular component, functional group, atom, etc.

The term “derivative”, refers to compounds which are formed from a parent compound by one or more chemical reaction(s).

The term “prodrug”, refers to an active drug chemically transformed into a per se inactive derivative which, by virtue of chemical or enzymatic attack, is converted to the parent drug within the body before or after reaching the site of action. Prodrugs are frequently (though not necessarily) pharmacologically inactive until converted to the parent drug. Methods for converting to drugs to prodrugs are known in the art. Suitable examples of prodrugs include, but are not limited to, ester and amide prodrugs; polyethylene glycol prodrugs with or without a linker; carbonate prodrugs; and dihydroxypropyl prodrugs. “Pharmaceutically acceptable salt”, refers to the modification of the parent compound by making the acid or base salts thereof. Examples of pharmaceutically acceptable salts include, but are not limited to, mineral or organic acid salts of basic residues such as amines and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

### II. Compositions

A. 3α-hydroxy-5α-pregnan-20-one (APα)

The compositions contain one or more neuro-enhancing agents. In one embodiment, the one or more neuro-enhancing agents are selected from progesterone or an analogue or derivative thereof, such as precursors of progesterone, progesterone metabolites and progesterone derivatives in its metabolic pathway, as well as the salts or hydrates of these analogues and derivatives.

In a preferred embodiment, the compositions contains a naturally occurring metabolite of progesterone, 3α-hydroxy-5α-pregnan-20-one (Allo; APα), also known as tetrahydroprogesterone (THP), as well as the pharmaceutically acceptable salts and hydrates thereof. 3α-hydroxy-5α-pregnan-20-one (THP) is generally classified as a neurosteroid as it is produced in the central nervous system and previously has been found to be an allosteric modulator of GABA receptors.

Other suitable analogs and derivatives include variant molecules of 3α-hydroxy-5α-pregnan-20-one or substituted derivatives of 3α-hydroxy-5α-pregnan-20-one, such as 3α-oxy derivatives, 3α-alkyl derivatives, 3α-alkenyl derivatives, 3α-ester derivatives, 3α-ether derivatives; 3ss-phenylethynyl derivatives of 3α-hydroxy-5α-pregnan-20-one, and 3p-phenylethynyl derivatives of 3α-hydroxy-5α-pregnan-20-one, as described in Hawkinson, et al. J. Pharmacology & Experimental Therapeutics 287: 198-207 (1998); as well as steroids derivatives of the 5α pregnan-20-one series such as those described in U.S. Pat. Nos. 5,925,630; 6,143,736; and 6,277, 838.

Analogs or derivatives of 3α-hydroxy-5α-pregnan-20-one include progesterone-like molecules that are either natural precursors or metabolites of progesterone or synthetic variants of progesterone that exhibit substantially equivalent neurogenic activity as 3α-hydroxy-5α-pregnan-20-one. Substantially equivalent neuro-enhancing activity is defined as approximately 30% to approximately 300% of the neuro-enhancing activity of 3α-hydroxy-5α-pregnan-20-one.

The agents are administered at dosages and for periods of time effective to stimulate or induce neural proliferation and/or to protect against neural loss in an individual. It has been established that a dosage of between about 2 mg and about 10 mg Allo is optimally effective for treating or preventing one or more symptoms of Alzheimer's disease or dementia in a human. For example, the dosage of the Allo is in the range of about 2 mg to about 10 mg, more preferably in the range of about 3 mg to about 5 mg, more preferably in the range between about 3.5 mg and about 4.5 mg, most preferably 4 mg.

The compounds may have one or more chiral centers and thus exist as one or more stereoisomers. Such stereoisomers can exist as a single enantiomer, a mixture of diastereomers or a racemic mixture. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

B. Additional Active Agents

The compositions can further contain one or more additional active agents. In one embodiment, the additional active agent is a steroid. Suitable steroids include biologically active forms of vitamin D3 and D2, such as those described in U.S. Pat. Nos. 4,897,388 and 5,939,407. The steroids may be co-administered to further aid in neurogenic stimulation or induction and/or prevention of neural loss, particularly for treatments of Alzheimer's disease. Estrogen and estrogen related molecules also may be co-administered with the neuro-enhancing agents to enhance neuroprotection as described in Brinton (2001) Learning and Memory 8 (3): 121-133.

Other neuroactive steroids, such as various forms of dehydroepi-androsterone (DHEA) as described in U.S. Pat. No. 6,552,010, can also be co-administered to further aid in neurogenic stimulation or induction and/or prevention of neural loss. Other agents that cause neural growth and outgrowth of neural networks, such as Nerve Growth Factor (NGF) and Brain-derived Neurotrophic Factor (BDNF), can be administered either simultaneously with or before or after the administration of THP. Additionally, inhibitors of neural apoptosis, such as inhibitors of calpains and caspases and other cell death mechanisms, such as necrosis, can be co-administered with the neuro-enhancing agents to further prevent neural loss associated with certain neurological diseases and neurological defects.

C. Formulations

Depending upon the manner of introduction, the neuro-enhancing agents may be formulated in a variety of ways. Formulations containing THP or other substantially equivalent variant molecules can be prepared in various pharmaceutical forms, such as granules, tablets, capsules, suppositories, powders, controlled release formulations, suspensions, emulsions, creams, gels, ointments, salves, lotions, or aerosols and the like.

In one embodiment, the neuro-enhancing agent are formulated as solid dosage forms suitable for simple, and preferably oral, administration of precise dosages. Solid dosage forms for oral administration include, but are not limited to, tablets, soft or hard gelatin or non-gelatin capsules, and caplets. However, liquid dosage forms, such as solutions, syrups, suspension, shakes, etc. can also be utilized.

In another embodiment, the formulation is administered topically or transdermally. Suitable topical and transdermal formulations include, but are not limited to, lotions, ointments, creams, and gels. In a preferred embodiment, the transdermal formulation is a gel. “Topical”, as used herein, generally refers to formulations for local delivery of an active agent, for example, via a gel, lotion, cream, ointment, or patch. “Transdermal”, as used herein, generally refers to systemic delivery of a drug through the unbroken skin, for example, via a gel, lotion, cream, ointment, or patch.

In another embodiment, the formulation is administered intranasally. Examples of intranasal formulations include aqueous preparations, preparations containing one or more inhalants, and dry powder formulations. The nasal mucosa is highly vascularized; the delivery of a thin layer of medication across a broad surface area can result in rapid transmucosal absorption of the medication into the blood stream and cerebral spinal fluid. This can result in more rapid achievement of therapeutic drug levels compared to oral or parenteral formulations.

Formulations containing one or more of the compounds may be prepared using a pharmaceutically acceptable carrier composed of materials that are considered safe and effective and may be administered to an individual without causing undesirable biological side effects or unwanted interactions. The carrier is all components present in the pharmaceutical formulation other than the active ingredient or ingredients. As generally used herein “carrier” includes, but is not limited to, diluents, binders, lubricants, disintegrators, fillers, pH modifying agents, preservatives, antioxidants, solubility enhancers, and coating compositions.

Carrier also includes all components of coating compositions which may include plasticizers, pigments, colorants, stabilizing agents, and glidants. Delayed release, extended release, and/or pulsatile release dosage formulations may be prepared as described in standard references known in the art. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The proportion of pharmaceutically active neuro-enhancing agent to carrier and/or other substances may vary between about 0.5 and about 100 wt % (weight percent). For oral use, the pharmaceutical formulation will generally contain between about 5 and about 100% by weight of the active material. For other uses, the pharmaceutical formulation will generally have between about 0.5 and about 50 wt. % of the active material.

1. Transdermal Formulations

Suitable transdermal formulations include lotions, ointments, creams, gels, and patches. A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers. A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes. Self generating emulsions are known to enhance the absorption of drugs as shown in the following table.

In one embodiment, the formulation is a transdermal gel. A “gel” is a semisolid system containing a dispersion of the active agent, i.e., allopregnanolone, in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. “Lipophilic” refers to compounds having an affinity for lipids.

The gelling agent can be natural, semi-synthetic, or synthetic. Suitable thickening or gelling agents include, but are not limited to, acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C₉-C₁₅ alcohols, calcium acetate, calcium alginate, calcium carrageenan, calcium chloride, caprylic alcohol, vinyl polymers such as cross linked acrylic acid polymers with the name carbomer, such as but not limited to carbomer 910, carbomer 934, carbomer 934P, carbomer 940, carbomer 941, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M is also known as Polyox WSR® N-IO, which is available from Union Carbide and as PEG-2,000; PEG-5M is also known as Polyox WSR® N-35 and Polyox WSR® N-80, both available from Union Carbide and as PEG-5,000 and Polyethylene Glycol 300,000; PEG-7M is also known as Polyox WSR® N-750 available from Union Carbide; PEG 9-M is also known as Polyox WSR® N-3333 available from Union Carbide; PEG-14M is also known as Polyox WSR® N-3000 available from Union Carbide., polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacrylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof.

The concentration of gelling agent can be adjusted to change the viscosity of the gel. For example, in some embodiments the formulation includes 10%, 20%, 30%, 40%, 50%, 60%, or 70% w/v of a gelling agent. Alternatively, the gelling agent can be in a range of 1-80% w/v.

Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug.

The concentration of the solvent can also be adjusted. For example, in some embodiments the formulation includes 10%, 20%, 30%, 40%, or 50% v/v of solvent. Alternatively, the solvent can be in a range of 1-50% v/v.

The gel may contain one or more penetration enhancers, for example to cross the barrier of the stratum corneum. Suitable enhancer include, but are not limited to, urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

The gel may also contain a preservative. Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

Transdermal formulations can be prepared to provide sustained or extended release of the neuro-enhancing agents.

In a preferred embodiment, the gel contains ethanol as a solvent and carbomer 940 as the gelling agent.

2. Intranasal Formulations

In one embodiment, the compounds are formulated for intranasal administration for delivery of the compounds to the brain. The olfactory mucosa are in direct contact with the brain and CSF. Therefore, medications absorbed across the olfactory mucosa directly enter the CSF. This provides a rapid, direct route for drug delivery to the brain. Bioavailability for drugs can be much higher when administered intranasally versus other routes of administration. Further, intranasal administration avoids the gut thereby bypassing first pass metabolism by the liver.

The compounds can be formulated as solutions or suspensions in an aqueous or organic solvent or as a dry powder. For suspensions and dry powder formulations, particles sizes of 10-50 microns adhere best to the nasal mucosa, as smaller particles may pass on to the lungs and larger particles can form droplets and run out of the nose. Atomized drugs are typically more effective than liquids since they provide larger surface area coverage and the smaller particle size provides a thin layer to cover the mucosa.

Compounds can be administered intranasally in the form of drops which are administered using a syringe or dropper, sprays or atomized formulations which provide a unit dose, such as a via syringe or a unit dose pump, or nebulized formulations. Devices for administering drugs intranasally are well known in the art.

Intranasal formulations may contain one or more excipients, such as penetration enhancers, surfactants, preservatives, etc.

3. Enteral Formulations

Pharmaceutical compositions for oral administration can be liquid or solid. Liquid dosage forms suitable for oral administration include, but are not limited to, pharmaceutically acceptable emulsions, microemulsions, solutions, suspensions, syrups and elixirs. In addition to an encapsulated or unencapsulated HDAC inhibitor, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor and sesame oils), glycerol, tetrahydrofuryl alcohol, polyethylene glycols and fatty acid esters of sorbitan and mixtures thereof. Besides inert diluents, the oral compositions can also include adjuvants, wetting agents, emulsifying and suspending agents, sweetening, flavoring and perfuming agents.

Solid dosage forms for oral administration include, but are not limited to, capsules, tablets, caplets, dragees, powders and granules. In such solid dosage forms, the encapsulated or unencapsulated compound is typically mixed with at least one inert, pharmaceutically acceptable excipient or carrier such as sodium citrate or dicalcium phosphate and/or (a) fillers or extenders such as starches, lactose, sucrose, glucose, mannitol and silicic acid, (b) binders such as, for example, carboxymethylcellulose, alginates, gelatin, polyvinylpyrrolidinone, sucrose and acacia, (c) humectants such as glycerol, (d) disintegrating agents such as agar-agar, calcium carbonate, potato or tapioca starch, alginic acid, certain silicates and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay and (i) lubricants such as talc, calcium stearate, magnesium stearate, solid polyethylene glycols, sodium lauryl sulfate and mixtures thereof. In the case of capsules, tablets and pills, the dosage form may also contain buffering agents.

Solid compositions of a similar type may also be employed as fill materials in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art.

4. Modified Release Formulations

The compositions can be formulation for modified or controlled release. Examples of controlled release dosage forms include extended release dosage forms, delayed release dosage forms, pulsatile release dosage forms, and combinations thereof.

### III. Methods of Use

Methods for reducing, preventing, or reversing the learning and/or memory deficits in an individual suffering from Alzheimer's disease are provided. The methods include administering an effective amount of the composition of 3α-hydroxy-5α-pregnan-20-one, or a derivative or analog, or pharmaceutically acceptable salt thereof to the subject in an amount between about 2 mg and about 6 mg. The methods include repeating the administration once every 7 days, or less frequently. Typically, a single dose of from 2 mg to 6 mg Allo is administered once within a 24 hour period, and the dosing is repeated once a week, or less frequently. In some embodiments, a single dose of from 2 mg to 6 mg Allo is administered repeatedly. Typically, the dose is administered once within 7 days, or less frequently, for a period of between one month and one year. In some embodiments, the composition is repeatedly administered once per week or less frequently for a period of three months, six months, 9 months, one year, or more than one year.

Methods including administering Allo in an amount of from 2 mg to 6 mg to the subject for a period of time effective to reduce the amount of (3-amyloid protein in the brain of the subject are provided. The amount of in the Allo administered in a single dose within a single 7 day period is between about 2 mg and about 6 mg, preferably between about 3 and about 5 mg, more preferably about 4 mg.

In an exemplary embodiment, a dosage of 4 mg Allo is administered via systemic route, such as by intravenous, or by transdermal administration, in a manner such that the entire dosage of 4 mg is administered to the subject within a period of 1-2 hours. Administration of the same dosage of Allo is repeated once or more times to the same subject after a period of at least 6 days, such as after 7 days, after 8 days, after 9 days, or after more than 9 days. In some embodiments a dosage regimen “cycle” includes administering a first dose of an amount of Allo between about 2 mg and about 6 mg on day 1, then no dose on day 2, no dose on day 3, no dose on day 4, no dose on day 5, no dose on day 6, no dose on day 7. A second cycle includes administering a second dose of Allo between about 2 mg and about 6 mg on day 8, then no dose on day 9, no dose on day 10, no dose on day 11, no dose on day 12, no dose on day 13, and no dose on day 14. This regimen is repeated for as many cycles as is deemed effective to treat one or more symptoms of AD, or to prevent or delay the onset of one or more symptoms of AD.

The compositions provide an effective amount of one or more neuro-enhancing agents upon administration to an individual. As used in this context, an “effective amount” of one or more neuro-enhancing agents is an amount that is effective to improve or ameliorate one or more symptoms associated with Alzheimer's disease, including neurological defects or cognitive decline or impairment. Such a therapeutic effect is generally observed within about 12 to about 24 weeks of initiating administration of a composition containing an effective amount of one or more neuro-enhancing agents, although the therapeutic effect may be observed in less than 12 weeks or greater than 24 weeks.

The individual is preferably an adult human, and more preferably the human is over the age of 30, who has lost some amount of neurological function as a result of Alzheimer's disease. Generally neural loss implies any neural loss at the cellular level, including loss in neurites, neural organization or neural networks. Examples of other subjects who can be treated include humans, dogs, cats, rats, and mice.

The compositions can be administered weekly, or less frequently in an amount to provide a therapeutically effective increase in the blood level of the one or more neuro-enhancing agents. For example, the total daily dosage will be about 3-5 mg and more preferably about 4 mg when administered systemically within a 24 hour period.

Where the administration is by other than an oral route, the neuro-enhancing agents or compositions may be delivered over a period of more than on hour, e.g., 3-10 hours, in an amount effective to produce a total dose of 4 mg within a 24 hour period. Alternatively, the compositions can be formulated for controlled release, wherein the composition is administered as a single dose that is repeated on a regimen of once a week, or less frequently.

In a preferred embodiment, the dosage of allopregnanolone is 4 mg administered once within a week. Therefore, a balance between optimal neurogenesis and optimal anti-amyloidogenic effects is predicted to be achievable with a once per week dosing schedule. The compositions are typically repeatedly administered for an extended period of time, for example, administered a total of 5-10 times over about 10 about weeks, a total of about 15-30 times over about 30 weeks, a total of 30-60 times over about 60 weeks, etc., and most preferably, administered regularly once per week or less frequently for as long as the patient is receiving noticeable benefit from the treatment method.

In a preferred embodiment, the composition containing one or more neuro-enhancing agents is administered to an individual as a single 4 mg dose, repeated once per week or less frequently for a period effective to produce an improvement in at least one criterion set forth as indicative of an improvement in one or more symptoms of Alzheimer's disease, including neurological defects or cognitive decline or impairment. Improvements include an improvement in cognitive abilities, memory, motor skills, learning or the like, preferably an improvement is observed in at least two such criteria.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the neuro-enhancing agents or compositions of the present invention. Following this initial assessment, treatment methods for the administration of the neuro-enhancing agents of the present invention are initiated and continued for various time intervals. At a selected time interval subsequent to the initial assessment of the neurological defect impairment, the same assessment or evaluation test (s) is again used to reassess changes or improvements in selected neurological criteria.

The compositions can be administered systemically in a variety of ways, such as orally, parenterally (e.g., subcutaneous, intravenous, intramuscular, transdermal, intraarterial, intraperitoneal, intrathecal, intracardiac, or intrasternal), transcutaneously, transmucosally, subcutaneously, by inhalation, infusion, particularly via intracerebroventricular infusion, although transdermal or intravenous administration is generally preferred. Depending on the route of administration, the compositions may be coated with or in a material to protect it from the natural conditions which may detrimentally affect its ability to perform its intended function. A particularly convenient method of administering compositions of the present invention is via transdermal administration. When administration is by way of a transdermal patch, the patch is applied to deliver a single dose within a 24 hour period. The patch is then removed and another patch is placed on the subject after a period of at least one week, to ensure dosing is not more than once per week. When a single transdermal patch is used to deliver multiple doses, the doses must be separated by a period of time of at least one week to achieve optimal efficacy. Continuous dosing, or dosing more frequently than once per week can lead to neurological decline.

A. Treatment of Alzheimer's Disease

Alzheimer's disease is an irreversible, progressive brain disease. It is characterized by the development of amyloid plaques and neurofibrillary, or tau, tangles; the loss of connections between nerve cells (neurons) in the brain; and the death of these nerve cells. There are two types of Alzheimer's—early-onset and late-onset. Both types have a genetic component. Early-onset Alzheimer's disease occurs between a person's 30s to mid-60s and represents less than 10 percent of all people with Alzheimer's. Some cases are caused by an inherited change in one of three genes, resulting in a type known as early-onset familial Alzheimer's disease, or FAD. For other cases of early-onset Alzheimer's, research suggests there may be a genetic component related to factors other than these three genes. Most people with Alzheimer's have the late-onset form of the disease, in which symptoms become apparent in the mid-60s and later. The causes of late-onset Alzheimer's are not yet completely understood, but they likely include a combination of genetic, environmental, and lifestyle factors that affect a person's risk for developing the disease.

Methods for treatment, reduction and prevention of the biological processes associated with Alzheimer's disease include administering Allo in an amount and dosing regimen effective to induce neuro-enhancement in a subject in need thereof. Neuro-enhancement resulting from the administration of compositions of Allo includes the stimulation or induction of neural mitosis leading to the generation of new neurons, i.e., exhibiting a neurogenic effect, prevention or retardation of neural loss, including a decrease in the rate of neural loss, i.e., exhibiting a neuroprotective effect, or one or more of these modes of action. The term “neuroprotective effect” is intended to include prevention, retardation, and/or termination of deterioration, impairment, or death of an individual's neurons, neurites and neural networks. Administration of the compositions leads to an improvement, or enhancement, of neurological function in an individual with a neurological disease, neurological injury, or age-related neuronal decline or impairment.

Neural deterioration can be the result of any condition which compromises neural function which is likely to lead to neural loss, Neural function can be compromised by, for example, altered biochemistry, physiology, or anatomy of a neuron, including its neurite. Deterioration of a neuron may include membrane, dendritic, or synaptic changes which are detrimental to normal neuronal functioning. The cause of the neuron deterioration, impairment, and/or death may be unknown. Alternatively, it may be the result of age-, injury- and/or disease-related neurological changes which occur in the nervous system of an individual.

In Alzheimer's patients, neural loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory.

Neural loss through disease, age-related decline or physical insult leads to neurological disease and impairment. The compositions can counteract the deleterious effects of neural loss by promoting development of new neurons, new neurites and/or neural connections, resulting in the neuroprotection of existing neural cells, neurites and/or neural connections, or one or more these processes. Thus, the neuro-enhancing properties of the compositions provide an effective strategy to generally reverse the neural loss associated with degenerative diseases, aging and physical injury or trauma.

Administration of between about 2 mg and about 10 mg of 3α-hydroxy-5α-pregnan-20-one, or a substantially equivalent variant molecule, to an individual who is undergoing or has undergone neural loss, as a result of Alzheimer's disease reduces any one or more of the symptoms of Alzheimer's disease, or associated cognitive disorders, including dementia. Clinical symptoms of AD or dementia that can be treated, reduced or prevented include clinical symptoms of mild AD, moderate AD, and/or sever AD or dementia.

In mild Alzheimer's disease, a person may seem to be healthy but has more and more trouble making sense of the world around him or her. The realization that something is wrong often comes gradually to the person and their family. Exemplary symptoms of mild Alzheimer's disease/mild dementia include, but are not limited to, memory loss; poor judgment leading to bad decisions; loss of spontaneity and sense of initiative; taking longer to complete normal daily tasks; repeating questions; trouble handling money and paying bills; wandering and getting lost; losing things or misplacing them in odd places; mood and personality changes, and increased anxiety and/or aggression.

Symptoms of moderate Alzheimer's disease/moderate dementia include, but are not limited to forgetfulness; increased memory loss and confusion; inability to learn new things; difficulty with language and problems with reading, writing, and working with numbers; difficulty organizing thoughts and thinking logically; shortened attention span; problems coping with new situations; difficulty carrying out multistep tasks, such as getting dressed; problems recognizing family and friends; hallucinations, delusions, and paranoia; impulsive behavior such as undressing at inappropriate times or places or using vulgar language; inappropriate outbursts of anger; restlessness, agitation, anxiety, tearfulness, wandering (especially in the late afternoon or evening); repetitive statements or movement, occasional muscle twitches.

Symptoms of severe Alzheimer's disease/severe dementia include, but are not limited to inability to communicate; weight loss; seizures; skin infections; difficulty swallowing; groaning, moaning, or grunting; increased sleeping; loss of bowel and bladder control.

Physiological symptoms of Alzheimer's disease/dementia include reduction in brain mass, for example, reduction in hippocampal volume. Therefore, in some embodiments, methods of administering Allo increase the hippocampal volume of the subject, reduce or prevent the rate of decrease of hippocampal volume, as compared to an untreated control subject.

B. Selection of Subjects for Treatment of Alzheimer's Disease

Methods for treatment or prevention of Alzheimer's disease can include selecting a subject in need thereof. The selection can include identifying a subject having one or more markers for Alzheimer's disease, such as a genetic predisposition. In some embodiments, the genetic marker is one or more alleles of the Apo-E4 gene. In other embodiments, the selection includes a clinical diagnosis of the patient. Exemplary clinical markers include the loss of brain mass and/or presence of β-amyloid within the brain of the subject. For example, in some embodiments the subject has lost up to 1%, more than 1%, more than 2%, up to 5%, or more than 5% of hippocampal mass within the past 12 months.

Therefore, methods for selecting a subject for treatment for Alzheimer's disease include screening the subject for the presence of the Apo-E4 gene allele, wherein the subject is selected for treatment if the subject carries the Apo-E4 gene allele. In some embodiments, a subject is selected for prophylactic treatment. For example, a subject identified as being at risk of developing one or more symptoms of Alzheimer's disease can be selected for prophylactic treatment in the absence of any clinical signs of Alzheimer's disease. A subject having no clinical symptoms, but having a genetic predisposition for Alzheimer's disease is a subject at risk of Alzheimer's disease. Therefore, in some embodiments the methods include identifying a subject having a genetic predisposition to Alzheimer's disease, and treating the subject to prevent or delay the onset, development or severity of Alzheimer's disease. Apolipoprotein E (ApoE) is a class of proteins involved in the metabolism of fats in the body. It is important in Alzheimer's disease and cardiovascular disease.

1. Apolipoprotein E/ApoE Gene

In some embodiments, methods for selecting a subject for treatment for Alzheimer's disease include screening the subject for the presence of the Apo-E4 gene allele.

Lipoproteins are molecules composed of fats and proteins. Apolipoprotein E is a fat-binding protein (apolipoprotein) that is part of the chylomicron and Intermediate-density lipoprotein (IDLs). These are essential for the normal processing (catabolism) of triglyceride-rich lipoproteins. In peripheral tissues, ApoE is primarily produced by the liver and macrophages, and mediates cholesterol metabolism. In the central nervous system, ApoE is mainly produced by astrocytes and transports cholesterol to neurons via ApoE receptors, which are members of the low density lipoprotein receptor gene family. ApoE is the principal cholesterol carrier in the brain.

Having one form of the apolipoprotein E (APOE) gene on chromosome 19 does increase a persons risk for developing AD within their lifetime. APOE comes in several different forms, or alleles. APOE ε2 is relatively rare and may provide some protection against the disease. If Alzheimer's disease occurs in a person with this allele, it usually develops later in life than it would in someone with the APOE ε4 gene. APOE ε3, the most common allele, is believed to play a neutral role in the disease—neither decreasing nor increasing risk. APOE ε4 increases risk for Alzheimer's disease and is also associated with an earlier age of disease onset. A person has zero, one, or two APOE ε4 alleles. Having more APOE ε4 alleles increases the risk of developing Alzheimer's. APOE ε4 is called a risk-factor gene because it increases a persons risk of developing the disease.

Methods for identifying a subject having a genetic predisposition to develop Alzheimer's disease include identifying which APOE alleles the subject has. Therefore, methods of identifying a subject at risk of AD can include genetic testing. Exemplary methods for genetic screening of a subject for the presence of a specific genotype are known in the art, including whole genome sequencing, or partial genome sequencing, for example, sequencing of chromosome 19.

### IV. Kits

The compositions can be packaged in kit. The kit can include a single dose or a plurality of doses of a composition containing one or more neuro-enhancing agents, and instructions for administering the compositions. Specifically, the instructions direct that an effective amount of the composition be administered to an individual with a particular neurological disease, defect or impairment as indicated. The composition can be formulated as described above with reference to a particular treatment method and can be packaged in any convenient manner

In an exemplary embodiment, a kit includes one or more dosages of form 2 mg to 10 mg of Allo, or a derivative, or analog, or pharmaceutically active salt thereof, and means for administration. In some embodiments, the kit includes one or more syringes, each syringe including a single dose of Allo for injection. The kit may also include one or more means for determining the presence of a genetic marker, such as a kit for detecting the presence of the ApoE4 allele.

Typically, kits include instructions for administering a single dose of from 2 mg to 10 mg Allo once per week or less frequently. The instructions can be affixed to the packaging material or can be included as a package insert. While the instructions typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions. Embodiments of the present invention also include the use of the above-described pharmaceutical products for the treatment of a human patient with a neurological disease, neurological defect or age-related neurological decline or impairment.

The following examples are put forth so as to provide those of ordinary skill in the art with a complete disclosure of how to make, to use and to evaluate the therapeutic agents, compositions and methods of the present invention, and are not intended to limit the scope of what is regarded as the invention. Efforts have been made to ensure accuracy with respect to numbers presented (e, g, amounts, concentrations, etc.), but some experimental errors and deviations should be allowed for.

### EXAMPLES

### Example 1. Clinical Trials to Determine Effects of 3α-Hydroxy-5α-Pregnan-20-One (THP) on Hippocampal Neural Cells in Adult Human Subjects with AD

Allopregnanolone (Allo) is a first in class regenerative therapeutic for delaying progression and treating Alzheimer's disease (AD) with a well characterized mechanism of action, preclinical evidence of efficacy and human safety.

### Materials and Methods

Clinical Trials were carried out in adult humans (see gov Identifier: NCT02221622 for the ongoing Phase 1b/2a clinical trial at https://clinicaltrials.gov/ct2/show/NCT02221622).

In both the central and peripheral nervous systems, Allo targets systems of regeneration and cholesterol trafficking in the brain to promote neurogenesis and cognitive function while simultaneously reducing the production of AD pathology or dementia and increasing indicators of white matter generation. Based on extensive preclinical discovery and translational research Allo was tested for activity in reducing and preventing symptoms of AD.

A randomized double-blind, placebo controlled, multiple ascending dose, phase 1 clinical trial was conducted in patients with mild cognitive impairment due to AD or mild AD or dementia.

Participants were age ≥55 years, had a MMSE score ≥20 and clinical dementia rating of 0.51. Participants were randomly assigned to receive weekly intravenous infusion of either 2 mg, 4 mg, or 6 mg of Allo or placebo (N=6/group) for 12 weeks. Primary outcomes were to assess safety, tolerability and determine maximally tolerated dose (MTD). Secondary exploratory outcomes were the feasibility and impact of Allo on MRI indicators of regeneration and cognition. Lymphocyte derived iPSCs differentiated to neural stem cells were used to develop biomarker strategy to identify potential regenerative responders.

The standard Phase I dose escalation double-blind, placebo-controlled, single site trial included 24 research participants in three dose cohorts. Each cohort included 8 research participants randomized to drug or placebo in a 6:2 allocation ratio. Cohorts as follows:
Dosing cohort 1) 2 mg Allo or placebo
Dosing cohort 2) 4 mg Allo or placebo
Dosing cohort 3) 6 mg Allo or placebo
   Each dose cohort was separated by 6 weeks to process, obtain, and assess all safety and tolerability data. Participants were observed for 30 days following the last dose for potential adverse events.

The trial evaluated three doses (2.0, 4.0 and 6.0 mg) of allopregnanolone (Allo) in a delivery vehicle of 6% sulfobutylether-β-cyclodextrin and 0.9% sodium chloride and placebo containing an equal volume of 0.9% sodium chloride for injection USP administered by intravenous sinfusion once per week for 12 weeks. Although the dosing study is not powered to detect statistically significant treatment differences on MRI outcomes and cognition, the MRI is required per FDA safety concerns with respect to amyloid-related imaging abnormalities, such as micro hemorrhages, as per FDA correspondence. Grouped differences from placebo-treated participants were evaluated in an exploratory fashion using the outcomes planned for the larger phase 2, proof of concept trial in order to obtain parameter estimates for the trial design.]

Allo dose selection of 2.0, 4.0 and 6.0 mg was determined by pre-clinical dose response analyses for neurogenesis, an IV bridging study in 3×TgAD mice, prior clinical studies, physiologically-based pharmacokinetics (PBPK) modeling and simulations of pharmacodynamic responses using available pre-clinical and clinical data of non-sedative to mildly sedative doses Planned study Allo doses are within the FDA requirement to not exceed 157 nmol/L (50 ng/ml) plasma concentration Planned Allo dose equivalencies based on 70 kg human are: 2.0 mg=0.028 mg/kg; 4.0 mg=0.056/mg/kg; 6.0 mg=0.084 mg/kg. With respect to safety, intravenous doses of Allo in the same dose range as those for this trial have been administered previously with acceptable safety and. In those studies TEAEs were mild and transient and included nausea, mild sedation, fatigue, flushing, and anxiety (Table 1). Allo in sulfobutylether-β-cyclodextrin and 0.9% sodium chloride formulation was administered intravenously over 30 minutes once per week for 12 weeks. Participants were monitored under medical supervision for a minimum of two hours after each dose. The regimen for Allo administration is based on preclinical analyses that indicate that a once per week treatment regimen results in increased neurogenesis with reduction in AD pathology.

### Results

Trial participants were recruited from the ultimate target population of persons diagnosed with either mild cognitive impairment (MCI) due to AD, early AD or dementia. Twenty-four participants were equally distributed to include 12 postmenopausal women and 12 men, 55 years of age or older, with a MMSE >20, who retain capacity to provide informed consent.

Based on these criteria, twenty-four AD patients were enrolled into the trial (18 Allopregnanolone/6 placebo). Peak plasma levels were reached within 30 minutes of start of infusion. Mean Cmax at 2 mg, 4 mg and 6 mg was 63±21 nM, 130±26 nM and 248±84 nM, respectively. Cmax closely correlated (R=0.77) with Allo delivered in mg/kg dose. MTD was established by onset of sedation at doses >6 mg. Twelve week exposure to multiple doses of Allo once per week resulted in no reportable adverse effects, serious adverse events or ARIA. Structural analysis of MRI based indicators of gray matter volume were consistent with regeneration in select brain regions. Subfield analysis indicated an increase in left hippocampal volume in the Allo 4 mg cohort. Cognitive function measured by ADAScog14 was not improved. However, some Cog state indicators were consistent with improvement. Biomarker of Allo response correlated with change in MRI structural volume.

The experimental data demonstrate that administration of Allo increased brain mass in human subjects over 55 years of age having a genetic marker associated with Alzheimer's diseases (Apo E4). Specifically, data indicated that patients having one or more alleles of ApoE4 gene (e.g., Apo-E 3/4, Apo-E 4/4, etc.) benefit from reduction in AD-related symptoms following administration of Allo.

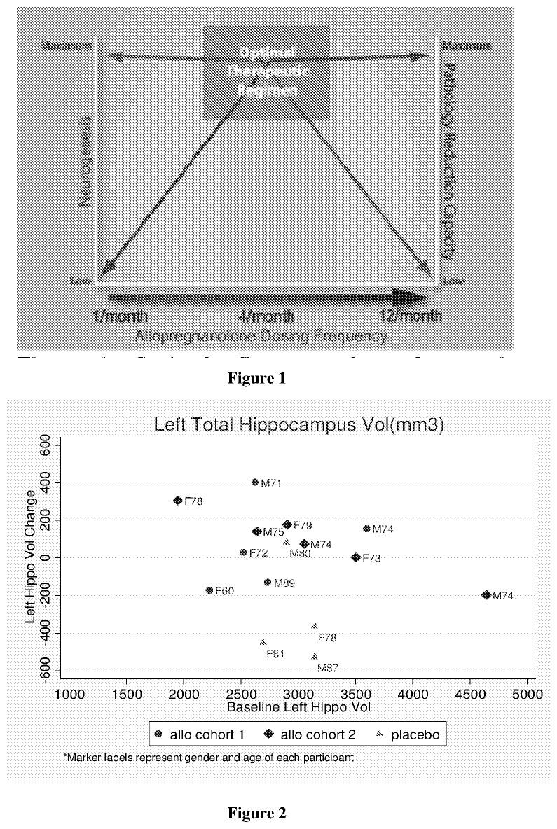

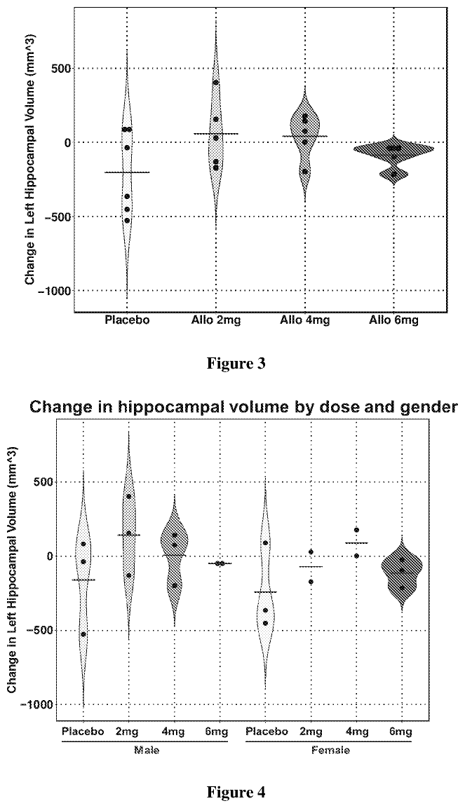

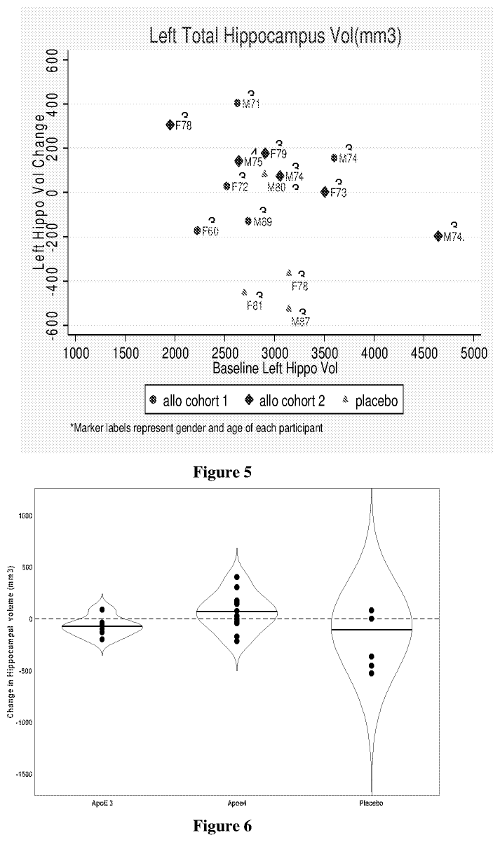

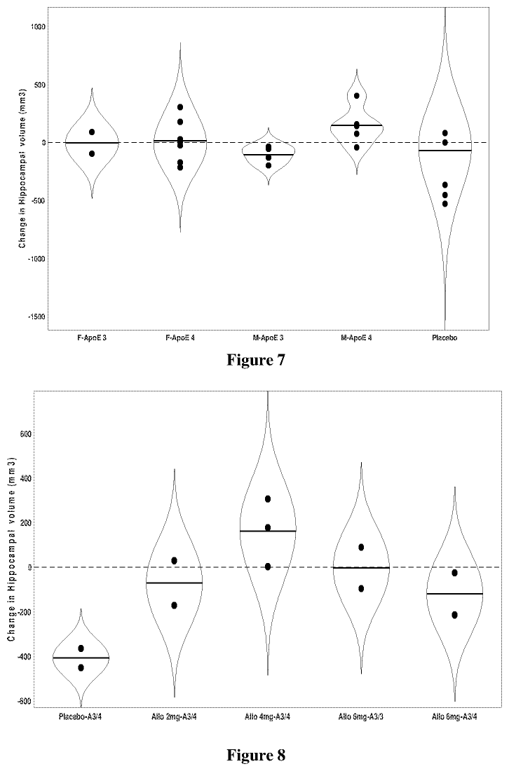

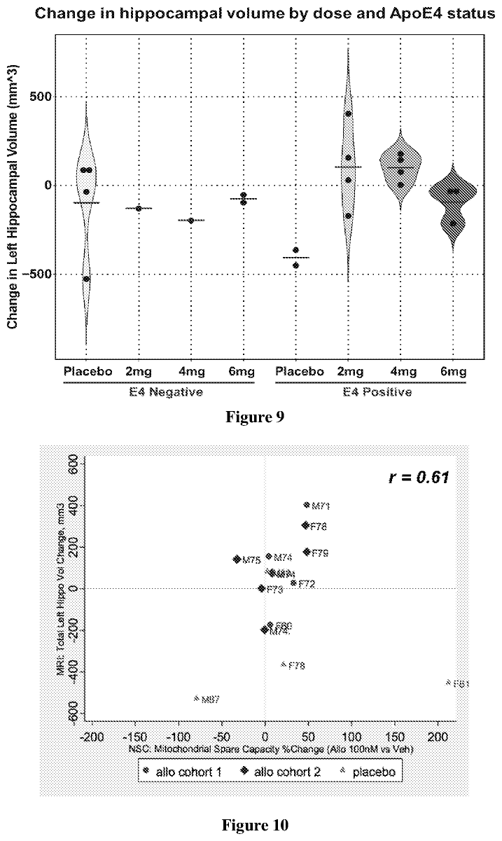

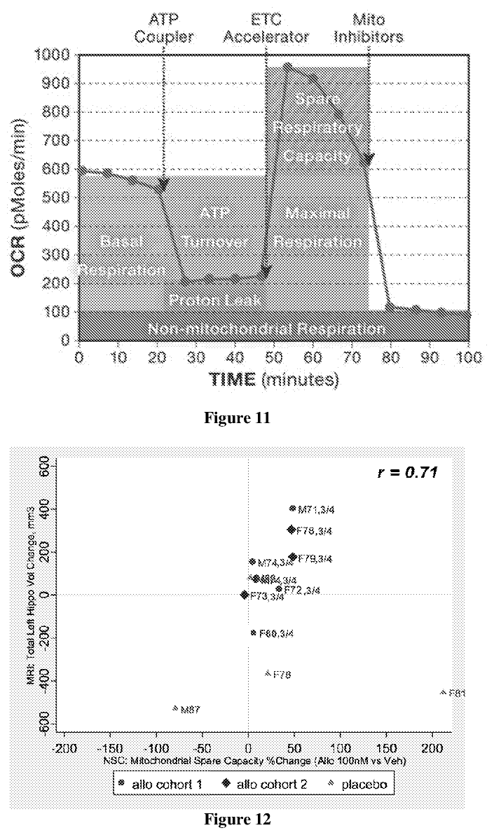

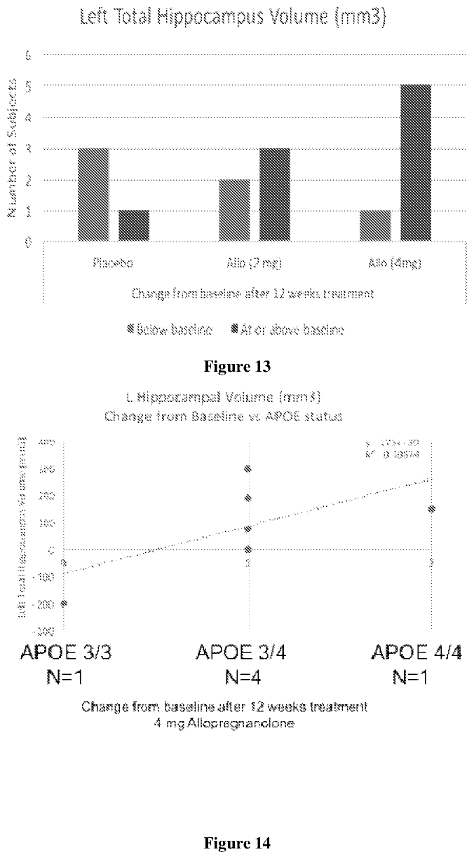

The results are presented in FIGS. 1-14.

### Example 2: Effect of Allopregnanolone on Memory Function in Humanized ApoE3,3, ApoE4,4 and Apo E3,4 Mice

### Materials and Methods

To access impact of Allopregnanolone (Allo) on memory function, humanized ApoE3,3, ApoE4,4 and ApoE3,4 mice were tested using Novel Object Recognition (NOR) in accordance with published literature (Antunes and Biala, Cogn Process. 2012 May; 13(2): 93-110; Ennaceur, Behav Brain Res. 2010 Dec. 31; 215(2):244-54. doi: 10.1016/j.bbr.2009.12.036. Epub 2010 Jan. 7; Leger et al., Nat Protoc. 2013 December; 8(12):2531-7. doi: 10.1038/nprot.2013.155. Epub 2013 Nov. 21; Piterkin et al., Learn. Mem., 15 (2008), pp. 785-791; Taglialatela et al., Behav Brain Res. 200(1): 95-99. 2009).

Behavioral NOR testing was conducted following 24 weekly injections of Allopregnanolone (Allo; 1.5 mg/mL) or saline (0.9%). The Allo and saline used to treat the mice were identical to the Allo and saline used in the clinical trial (ClinicalTrials.gov Identifier: NCT02221622).

Behavioral testing and analysis of behavioral outcomes were conducted blind to the experimental condition. Code for treatment groups was unblinded for statistical analyses by analyst who was independent of behavioral testing.

Difference in exploration time (in seconds) was calculated by subtracting the time spent exploring the familiar object from that of the novel object (Tn−Tf) and denoted by ‘Df’. Second, a ratio of the difference in exploration time to the total time spent exploring was calculated (Tn−Tf/Tn+Tf) and denoted by Discrimination Index (DI). The DI can vary between +1 and −1, where a positive score indicates more time spent with the novel object, a negative score indicates more time spent with the familiar object, and a zero score indicates a null preference (Antunes and Biala, 2012). The DI adjusts for difference in total exploration time (Broadbent et al.). Statistical analyses were conducted using Mann-Whitney test and p-value of less than 0.05 was considered significant.

### Results:

Allo exhibited a genotype specific responder effect in both females and males. Presence of the ApoE4 genotype was associated with increased cognitive function in Allo treated mice of both sexes. Allo significantly promoted memory and discrimination cognitive functions in ApoE4:4 females and males. In mice heterozygous ApoE3:4 female mice (male mice were not tested), Allo significantly increased memory function and trended towards a significant increased in discrimination function.

In ApoE3:3 mice, Allo had no effect either positive or negative on either cognitive function in either sex. These data support findings from an early phase human clinical trial of Allo in which a responder analysis of secondary outcomes indicated that ApoE4 carriers were responders to Allo whereas ApoE3:3 carriers were not (ClinicalTrials.gov Identifier: NCT02221622).

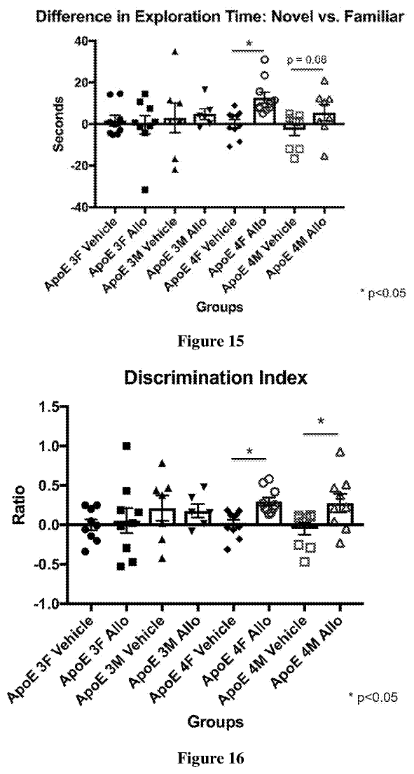

FIG. 15 shows the impact of allopregnanolone treatment on Novel Object Exploration time in humanized ApoE3:3 and ApoE4:4 female and male mice. In ApoE3:3 females and males, Allo treatment had no effect, either positive or negative, on exploration time. In contrast, Allo significantly increased exploration time in ApoE 4:4 females and trended towards an increase in ApoE4:4 males compared to their saline-treated counterparts.

As shown in FIG. 16, impact of allopregnanolone (Allo) treatment on Novel Object Discrimination Index in humanized ApoE3:3 and ApoE4:4 female and male mice, in ApoE3:3 females and males, Allo treatment had no effect on discrimination index. In contrast, Allo significantly increased the discrimination index of ApoE 4/4 females and males compared to their saline-treated counterparts (* p<0.05).

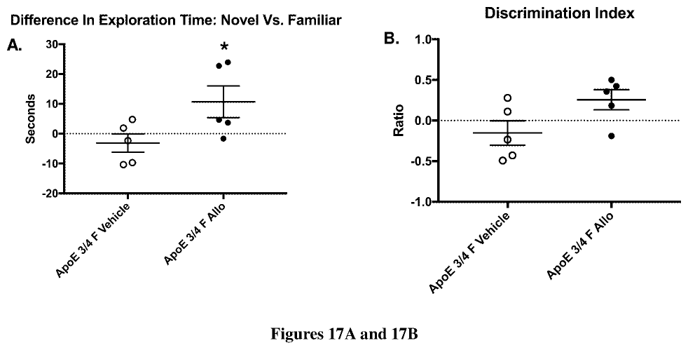

As shown in FIGS. 17A and 17B, impact of allopregnanolone (Allo) treatment on Novel Object Exploration and Discrimination Index in humanized ApoE3:4 female mice, allo treatment significantly increased exploration of the novel object in ApoE 3:4 female mice (p=0.05) and trended towards a significant increase in ability to discriminate between novel vs familiar object (p=0.06).

In summary, Allo significantly increased exploration of novel objects in ApoE4:4 mice indicating increased memory of the object to which the ApoE4:4 mouse was previously exposed (FIG. 15). Allo treated ApoE4:4 females achieved statistically significant novel object exploration whereas Allo treated males exhibited a trend towards a significant increase in exploration (FIG. 15). Allo treatment significantly increased the ability to discriminate the novel from the familiar object in both ApoE 4:4 females and males compared to their saline-treated counterparts (FIG. 16). Allo effect on discrimination index was specific to ApoE4:4 mice with no effect in ApoE3:3 females and males (FIGS. 17A and 17B).

In mice heterozygous for the ApoE4 gene, ApoE3:4 carriers, Allo significantly increased memory, exploration of novel object, and trended towards significant increase in discrimination function.

Collectively, these data demonstrate an ApoE4 genotype specific effect of Allopregnanolone and provide translational evidence in support of a ApoE4 genotype specific effect of Allo to promote cognitive function.

All patent and non-patent references cited in this specification are herein incorporated by reference as if each individual patent or non-patent reference were specifically and individually indicated to be incorporated by reference.

Although the foregoing embodiments of the invention has been described in some detail by way of illustration and example for purposes of clarity and understanding, it will be readily apparent to those of ordinary skill in the art in light of the teachings of this invention that certain changes and modifications may be made thereto without departing from the scope of the appended claims.

Those skilled in the art will recognize, or be able to ascertain using no more than routine experimentation, many equivalents to the specific embodiments of the invention described herein. Such equivalents are intended to be encompassed by the following claims.

1. A dosage formulation for systemic administration to a human with Alzheimer's disease or dementia, in a dosing regimen of no more frequently than a period of 24 hours or less systemic levels over a period of more than six days, the dosage formulation comprising a compound selected from the group consisting of 3a-hydroxy-5a-pregnan-20-one, a derivative or analog thereof, or a pharmaceutically acceptable salt thereof, in an amount between about 2 mg and about 10 mg, inclusive.

2. The dosage formulation of claim 1, wherein the compound is 3a-hydroxy-5a-pregnan-20-one.

3. The dosage formulation of claim 1, formulated for injection.

4. The dosage formulation of claim 1 in a formulation for oral administration.

5. The dosage formulation of claim 1 comprising a gel.

6. The dosage formulation of claim 5, wherein the gel comprises a thickening agent, solvent and/or transdermal penetration enhancer.

7. The dosage formulation of claim 6, wherein the thickening agent is a cross linked acrylic acid polymer.

8. The dosage formulation of claim 1, wherein the compound is present in an amount effective to reverse the learning and/or memory deficits in an individual suffering from Alzheimer's disease.

9. The dosage formulation of claim 1 wherein the compound is present in an amount effective to reduce β-amyloid expression.

10. The dosage formulation of claim 1 wherein the compound is a neuro-enhancing agent present in the dosage formulation is 4 mg.

11. The dosage formulation of claim 1, wherein the dosage of the agent is between 0.01 mg/kg and about 0.08 mg/kg body weight of a normal adult human having an average weight of 60-70 kg.

12. A method for reducing, preventing, or reversing the learning and/or memory deficits in an individual suffering from Alzheimer's disease or dementia, comprising administering an effective amount of the dosage formulation of claim 1,
wherein the composition is administered once within a 24 hour period, and
wherein the dosing is repeated once every 7 days, 8 days, 9 days, or a longer period of time.

13. The method of claim 12, wherein the dosage formulation is administered over a period of between one month and one year.

14. The method of claim 13, wherein the dosage formulation is administered for a period of more than one year.

15. The method of claim 13, wherein the dosage formulation is administered for a period of at least six months.

16. The method of claim 12, wherein the dosage formulation is administered for a period of time effective to reduce the amount of β-amyloid protein in the brain of the subject.

17. The method of claim 12, wherein the amount of 3α-hydroxy-5α-pregnan-20-one or a derivative or analog thereof in the dosage formulation is between about 2 mg and about 6 mg, preferably between about 3 and about 5 mg, more preferably about 4 mg.

18. The method of claim 12, wherein the amount of 3α-hydroxy-5α-pregnan-20-one or a derivative or analog thereof is 4 mg.

19. The method of claim 12, wherein the patient carries the Apolipoprotein E gene ε4 allele.

20. The method of claim 12, wherein the individual has lost more than 5% of their hippocampal mass within the past 12 months.

21. The method of claim 12, wherein the individual is a female human.

22. A method for selecting a human subject for treatment for Alzheimer's disease with the dosage formulation of claim 1, the method comprising the step of
(a) screening the individual for the presence of the Apo-E4 gene allele,
wherein the individual is selected for treatment if the subject carries the Apo-E4 gene allele.

23. The method of claim 22, wherein the individual is female.

24. The method of claim 22, further comprising
the step of
(b) administering to the individual the dosage formulation of claim 1.

25. The method of claim 24, further comprising the step of
(c) repeating step (b) administering the dosage formulation within one day, no more than one day per seven day week.
